(19) United States
(12) Patent Application Publication
(10) Pub. No.: US 2025/0311967 A1
(43) Pub. Date: Oct. 9, 2025

(54) SYSTEM AND METHOD FOR EARLY IDENTIFICATION OF SKIN LESIONS

(71) Applicant: The Certification Collective, LLC, Mesa, AZ (US)

(72) Inventors: Gregory Seth Allen, Gilbert, AZ (US); Scott Moore, Mesa, AZ (US)

(21) Appl. No.: 19/170,934

(22) Filed: Apr. 4, 2025

Related U.S. Application Data
(60) Provisional application No. 63/575,539, filed on Apr. 5, 2024.

(51) Int. Cl. A61B 5/00

(52) U.S. Cl. CPC A61B 5/445; A61B 5/0077

(57) ABSTRACT

A method may include using a handheld device having a magnifying lens configured to produce a magnified image of an abnormal skin lesion. The magnifying lens comprises a vertical crosshair and a horizontal crosshair intersecting in a perpendicular configuration to segment the magnified image into quadrants, the crosshairs each having a linear scale disposed thereon. The method includes accessing qualities of a skin lesion observed in each quadrant.

### RELATED APPLICATIONS

This claims the benefit of U.S. Provisional Patent Application No. 63/575,539, filed Apr. 5, 2024, to Allen et. al., titled “System and Method for Early Identification of Skin Lesions,” the entirety of the disclosure of which is hereby incorporated by reference thereto.

### TECHNICAL FIELD

This document relates to a system for identifying abnormal skin lesions.

### BACKGROUND

Skin cancers are the most common form of cancer worldwide, with roughly 1.5 million new cases reported annually. Once detected and properly diagnosed, many common forms of skin cancer are highly treatable. However, many skin cancers are often underdiagnosed, overlooked, or missed altogether by primary care providers, or commonly treated within primary care settings rather than referred on to a more qualified specialist for follow-up. Early recognition and diagnosis are key in the effective management of skin cancer. Early detection of skin cancer saves lives in many cases, in particular with respect to diagnoses of melanoma. The ABCDE rule was introduced by a dermatologist named Dr. Bernard Ackerman, MD, as a method to identify clinical features of melanoma developing from moles. The ABCDE rule has been used extensively and has been very effective in saving countless lives.

Currently known methods, such as the ABCDE rule, for consistently recognizing and identifying elements that go beyond typical variations found in the skin for early detection of skin cancer are lacking and subjective. Some melanomas and many non-melanoma skin cancers may not exhibit the typical ABCDE patterns that can be identified using currently known methods, and as a result, these melanomas and non-melanomas may be missed during examination.

### SUMMARY

In some embodiments, a system for identifying a skin lesion includes using a handheld device with a magnifying lens to produce a magnified image of an abnormal skin lesion. The magnifying lens may include vertical and horizontal crosshairs intersecting in a perpendicular configuration. The crosshairs segment the magnified image into quadrants. Each crosshair may have a linear scale. The system includes placing the handheld device over an abnormal skin lesion of a patient. The intersection of the crosshairs may be located proximal to a center of the abnormal skin lesion.

In some embodiments, the system includes performing a first assessment. The first assessment may begin with identifying, within a first quadrant, an original border of the abnormal skin lesion. The assessment may include counting, within the first quadrant, each instance of a border of the abnormal skin lesion breaching the original order within the first quadrant. In some embodiments, the system includes repeating the first assessment for a second, a third, and a fourth quadrant.

In some embodiments, the system includes performing a second assessment. The second assessment includes counting, within the first quadrant, the number of color changes within the abnormal skin lesion as compared to typical skin color variations present in the surrounding skin. The second assessment may include recording the number of color changes within the first quadrant. The system may include repeating the second assessment for the second, third, and fourth quadrants. In some embodiments, the system includes performing a third assessment that includes counting, within the first quadrant, by way of direct contact with the abnormal skin lesion or through visual observation of the magnified image, the number of consistency changes in the abnormal skin lesion. In some embodiments, the system includes repeating the third assessment for the second, third, and fourth quadrants.

In some embodiments, the system includes calculating, for each quadrant, a total count resulting from the first, second, and third assessments. The system includes analyzing, for each quadrant, the severity of the abnormal skin lesion by comparing the total count in each quadrant with a rating scale. In some embodiments, the system includes assigning a rating to the abnormal skin lesion. The abnormal skin lesion may be assigned a rating as a whole. Based on the rating, the system may include recommending a course of action.

In some embodiments, the magnifying lens comprises a shape selected from the group consisting of a circular shape, an oval shape, an organic shape, a polygon shape, a rectangle shape, a square shape, and a geometric shape.

In some embodiments, the abnormal skin lesion may be selected from the group consisting of a basal cell carcinoma, a squamous cell carcinoma, a Merkel cell carcinoma, and a melanoma. The magnifying lens may have a magnification of between eight times to twelve times.

In some embodiments, when the total count is calculated as seven or greater, the abnormal skin lesion depicted within the quadrant is assigned a rating of severe. In some embodiments, when the total count is calculated to be between 5 and 6, the abnormal skin layer lesion depicted within the quadrant is assigned a rating of moderate. In some embodiments, when the total count is calculated to be between 3 and 4, the abnormal skin lesion depicted within the quadrant is assigned a rating of mild, and no immediate action is taken. In some embodiments, when the total count is calculated to be between 0 and 2, the abnormal skin lesion depicted within the quadrant is assigned a rating of normal, and no immediate action is taken. In some embodiments, when each of the quadrants has a total count of four or less, the abnormal skin lesion as a whole is assigned a rating of normal or mild. Based upon the assigned rating, a recommendation may be provided to the patient to monitor the lesion for changes and follow up with a medical professional within six months if changes are observed. In some embodiments, if changes are observed in the abnormal skin lesion during monitoring, the total count is increased, and the results are weighted. In some embodiments, when any one of the quadrants comprises a total count of five or greater, the abnormal skin lesion as a whole is assigned a rating of moderate or severe. In some embodiments, based upon the assigned rating, a recommendation is provided to the patient to consult a physician for a thorough examination. Based on the assigned rating, the physician may remove the abnormal skin lesion. In some embodiments, the system uses an image of the abnormal skin lesion.

In some embodiments, a method for identifying a skin lesion includes providing a device with a magnifying lens that is configured to produce a magnified image of an abnormal skin lesion. The magnifying lens may include vertical and horizontal crosshairs that intersect in a perpendicular configuration to segment the magnified image into quadrants. Each of the crosshairs may have a linear scale disposed thereon. The method includes placing the device over an abnormal skin lesion of a patient. In some embodiments, the method includes locating the intersection of the crosshairs proximal to the center of the abnormal skin lesion.

In some embodiments, the method includes performing a first assessment. The first assessment may include identifying, within a first quadrant, the original border of the abnormal skin lesion. The first assessment may also include counting within the first quadrant, each instance when a border of the abnormal skin lesion breaches the original border. In some embodiments, the method may also include recording the number of instances where the border of the abnormal skin lesion breaches the original border. The method may include repeating the first assessment for a second, third, and fourth quadrant.

The method may also include performing a second assessment. The second assessment may include counting, within the first quadrant, the number of color changes within the abnormal skin lesion as compared to typical skin color variations present in the surrounding skin. The second assessment may include recording the number of color changes. The method may include repeating the second assessment for the second, third, and fourth quadrants.

The method may include calculating, for each quadrant, a total count resulting from the first and the second assessments. The method may include analyzing, for each quadrant, the severity of the abnormal skin lesion by comparing the total count with a rating scale to assign a rating to each quadrant of the abnormal skin lesion. Based upon the assigned rating, a course of action may be recommended.

In some embodiments, the method uses a magnifying lens with a shape selected from the group consisting of a circular shape, an oval shape, an organic shape, a polygon shape, a rectangle shape, a square shape, and a geometric shape. In some embodiments, the method may also include performing a third assessment. The third assessment may include counting, within the first quadrant, by directly contacting the abnormal skin lesion or through visual observation of the magnified image, the number of consistency variations, or changes, in the abnormal skin lesion within the first quadrant. The third assessment may include calculating for the first quadrant a total count from the third assessment. The method may include repeating the third assessment for the second, third, and fourth quadrants.

In some embodiments, the total count from the third assessment is combined with the total count resulting from the first and second assessments. In some embodiments, the method includes analyzing, for each quadrant, the severity of the abnormal skin lesion by comparing the total count with a rating scale to assign a rating to each quadrant of the abnormal skin lesion. A course of recommended action may be given.

In some embodiments, the magnifying lens has a magnification between 8× and 12×. The linear scale of the magnifying lens may be configured to measure in millimeters. The method may include assigning a rating of severe to an abnormal skin lesion depicted within a quadrant when the total count is calculated as seven or greater. The method may also include assigning a rating of moderate to an abnormal skin lesion depicted within the quadrant when the total count for the quadrant is calculated as between 5 and 6. The method may include assigning a rating of mild to the abnormal skin lesion depicted within the quadrant when the total count for the quadrant is calculated as between 3 and 4. In some embodiments, the method may include assigning a rating of normal to an abnormal skin lesion depicted within the quadrant when the total count is calculated between zero and two. In some embodiments, the method is performed using an image of the abnormal skin. The method may also include assigning a rating of normal or mild to the abnormal skin lesion as a whole when each of the quadrants have a total count of four or less. The method may also include providing a recommendation to the patient to monitor the lesion for changes and follow up with a physician within six months if changes are observed based upon the assigned rating.

In some embodiments, when any one of the quadrants comprises a total count of five or greater, the abnormal skin lesion as a whole is assigned a rating of moderate or severe. A recommendation may be provided to the patient to consult a physician for a thorough examination based upon the assigned rating. The physician may remove the abnormal skin lesion.

In some embodiments, a handheld device includes a magnifying lens having a magnification of about 8× to 12×. The magnifying lens is configured to produce a magnified image of an abnormal skin lesion. The magnifying lens further comprises two crosshairs in a perpendicular configuration disposed thereon to segment the magnified image into equally sized quadrants. Each crosshair may have a linear scale disposed thereon. The linear scale is configured to be measured in millimeters. In some embodiments, the magnifying lens has a shape selected from the group of a circular shape, an oval shape, an organic shape, a polygon shape, a rectangle shape, a square shape, and a geometric shape.

In some embodiments, the magnifying lens comprises a reticle. The handheld device may include a housing that surrounds the magnifying lens. In some embodiments, the arc comprises a linear scale that is modified as a function of the magnification of the magnifying lens.

In some embodiments, the magnification linear scale may be adjusted using augmented reality to match that of the magnified images. The rating assigned to the abnormal skin lesion may be based on a numerical quantification.

The foregoing and other aspects, features, applications, and advantages will be apparent to those of ordinary skill in the art from the specification, drawings, and the claims. Unless specifically noted, it is intended that the words and phrases in the specification and the claims be given their plain, ordinary, and accustomed meaning to those of ordinary skill in the applicable arts. The inventors are fully aware that they can be their own lexicographer if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of.,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed aspects, it is intended that these aspects not be limited only to the specific structure, material or acts that are described in the preferred embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent from the DESCRIPTION and DRAWINGS, and from the CLAIMS.

### BRIEF DESCRIPTION OF THE DRAWINGS

Implementations will hereinafter be described in conjunction with the appended and/or included DRAWINGS.

FIG. 1 shows a plan view of a visual inspection tool according to some embodiments.

FIG. 2 shows a perspective view of the visual inspection tool shown in FIG. 1.

FIG. 3 shows a perspective view of a visual inspection tool according to some embodiments.

FIG. 4 shows a perspective view of a visual inspection tool according to some embodiments.

FIGS. 5A and 5B show schematic views of a patient's arm.

FIG. 6 shows a schematic view of a patient's back.

FIG. 7A shows a schematic of a patient's lesion viewed through a visual inspection device according to some embodiments.

FIG. 7B shows a schematic view of a medical professional performing a visual inspection method using a visual inspection tool according to some embodiments.

FIG. 8A shows an assessment record for use with a visual inspection method according to some embodiments.

FIG. 8B shows a schematic of a patient's lesion viewed through a visual inspection device according to some embodiments.

FIG. 9A shows an assessment record for use with a visual inspection method according to some embodiments.

FIG. 9B shows a schematic of a patient's lesion viewed through a visual inspection device according to some embodiments.

FIG. 10A shows an assessment record for use with a visual inspection method according to some embodiments.

FIG. 10B shows a schematic of a patient's lesion viewed through a visual inspection device according to some embodiments.

FIG. 11A shows an assessment record for use with a visual inspection method according to some embodiments.

FIG. 11B shows a schematic of a patient's lesion viewed through a visual inspection device according to some embodiments.

FIG. 12 shows an assessment record for use with a visual inspection method according to some embodiments.

FIG. 13 shows a completed assessment record for use with a visual inspection method according to some embodiments.

FIG. 14A shows a completed assessment record with a completed analysis for use with a visual inspection method according to some embodiments.

FIG. 14B shows an assessment scale for use with a visual inspection method according to some embodiments.

### DETAILED DESCRIPTION

The following detailed description provides numerous specific details. Those skilled in the relevant arts understand that embodiments of the disclosure may be practiced without these specific details. The disclosure may also be practiced in different and alternative configurations.

Unless specifically noted, it is intended that the words and phrases in the specification and the claims be given their plain, ordinary, and accustomed meaning to those of ordinary skill in the applicable arts. The singular forms “a,” “an,” and “the” include plural referents unless the context clearly dictates otherwise. Thus, for example, a reference to “a step” includes a reference to one or more of such steps. The words “exemplary,” “example,” “embodiment,” or various forms thereof are used herein to mean serving as an example, instance, or illustration. Any aspect or feature described herein as “exemplary” or as an “example” is not necessarily to be construed as preferred or advantageous over other aspects or designs. The examples are provided solely for purposes of clarity and understanding and do not limit or restrict the disclosure. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one.

The present disclosure is directed to a visual inspection tool (VIT) and methods of using the same. The VIT may aid in the analysis and early identification of skin lesions, and it is designed to be used with a visual inspection method (VIM). In some instances, the VIM may be known under the trademark or tradename “(ABC)² methodology” or “ABC² methodology.” The VIT (also known as an AllenLens™ or quadrascope) uses magnification and measurement marks along at least two crosshairs that divide the viewing area into quadrants. Dividing the viewing area into quadrants makes assessing the severity of any abnormal skin lesion simpler.

Advantageously, the system disclosed herein provides a mechanism by which a lesion may be numerically quantified by any person trained in the VIM methodology. Based on the numerical quantification, the VIM methodology can provide a recommendation to the patient. Data collected as part of the method is recorded in an assessment record. The assessment record, with reference to an analysis rating scale that is based on extensive clinical research, helps medical professionals recognize skin cancer earlier and increase the number of lives saved.

While the system is designed for use by physicians, it is equally designed for nurses, medical assistants, medical students, primary care physicians, and any member of the general public who is trained in the VIM methodology (collectively referred to herein as a “medical professional”) to increase the early detection of skin cancers.

In describing the implementations disclosed herein, the following terminology will be used in accordance with the definitions and explanations set out below. Notwithstanding, other terminology, definitions, and explanations may be found throughout this document as well.

As used herein, “lesion” is a term used in its broadest sense and may refer to any differentiation in the skin, ranging from a simple freckle or mole, to an injury/scarring, to a full basal cell carcinoma.

As used herein, “aerial” is a term used in its broadest sense and may refer to a quick, overall, distant view of the skin to look for anything on the skin that may appear different or look suspicious from the surrounding area.

As used herein, “abnormal” is a term used in its broadest sense and may refer to anything on the skin which looks different, new or changing than the surrounding area. An abnormality on the skin can include color differences, bumps, ulcers and other inconsistencies.

As used herein, “border” is a term used in its broadest sense and may refer to a perimeter or boundary encircling a skin lesion. A “well-defined” border around a skin lesion refers to a border that is clearly distinct and sharply defined from the surrounding skin. It may appear as a distinct, raised, or flat area with a smooth and uniform edge. In contrast, an ill-defined border around a skin lesion refers to a border that is not clearly separated from the surrounding skin. It may appear hazy, fuzzy, or smudged, with an uneven or irregular edge.

As used herein, a “breach” in a border is a term used in its broadest sense and may refer to a disruption or change in the edges or margins of a previously existing border of a skin lesion.

As used herein, “color change” is a term used in its broadest sense and may refer to a change or alteration in the hue, shade, or intensity of color of a particular area of the skin or a skin lesion compared to normal skin color.

As used herein, “consistency” is a term used in its broadest sense and may refer to the texture or feel of a skin lesion when observed or touched as compared to everything else on the baseline “normal” skin around it.

The present disclosure will now be described with reference to embodiments shown in the figures.

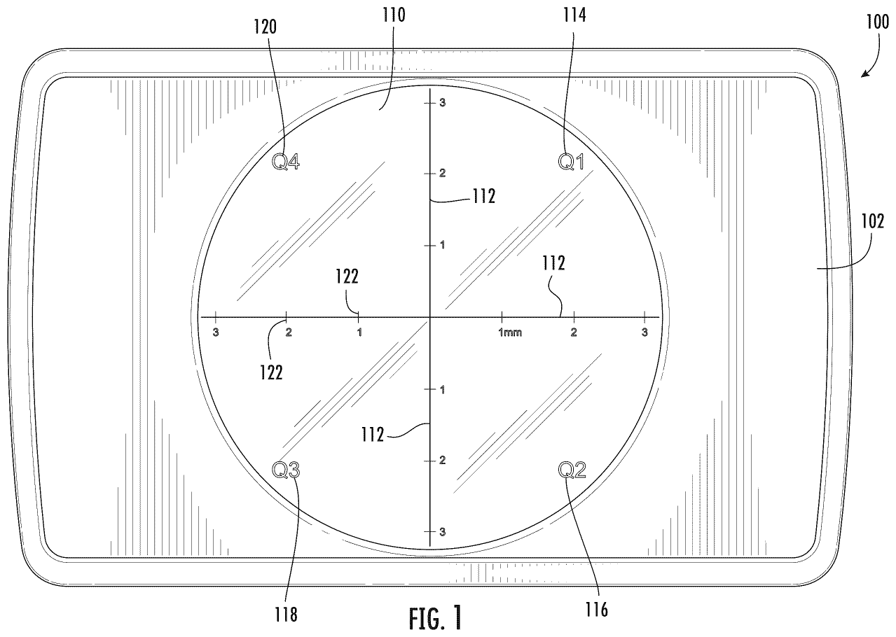

FIG. 1, shows a plan view of VIT 100, according to some embodiments. VIT 100 may be a handheld device and is configured to produce a magnified image of an abnormal skin lesion, such as, for example, a basal cell carcinoma, a squamous cell carcinoma, a Merkel cell carcinoma, a melanoma, and the like. VIT 100 includes magnifying lens 110 disposed in housing 102. The magnifying lens 110 or housing 102 may have any shape, for example, a circular shape, a polygon shape, a hexagon shape, a rectangle, a square shape, a chevron shape, a semi-circle, an oval, a pentagon shape, and a triangle shape, among others. Magnifying lens 110 and housing 102 may have any number of shapes. FIG. 1 shows rectangular housing 102 and circular magnifying lens 110. VIT 100 is preferably a portable, handheld device designed for ease of use by physicians during holistic skin examinations of patients.

In some embodiments, magnifying lens 110 may have a magnification from about 5×-20×, or from about 8×-12×, or about 10×. A person of ordinary skill in the art would understand that providing a magnified image of abnormal skin lesions of smaller size may require a higher power magnification, and any magnification for the lens may be used.

Magnifying lens 110 includes crosshairs 112. Two crosshairs 112 divide magnifying lens 110 into four quadrants: first quadrant 114, second quadrant 116, third quadrant 118, and fourth quadrant 120. Crosshairs 112 include reticles 122. Reticles 122 may be disposed on crosshairs 112 to represent a linear scale. The linear scale may be a function of the magnification of magnifying lens 110. For example, the linear scale may be marked as millimeters to correspond with the magnified image, yet an actual measurement of the linear scale would be increased by an amount relative to the magnification of magnifying lens 110. Stated another way, the linear scale as part of magnifying lens 110 is expanded to the same extent as magnifying lens 110 magnification such that a size of a feature measured using VIT 100 may be accurately approximated when VIT 100 is held at a focal length from the abnormal skin lesion where the magnified image is in best focus to the user. A linear scale that is a function of lens magnification improves the accuracy of measurements. Using a measurement device, such as a ruler or similar, placed directly on an abnormal lesion would be more difficult to measure accurately, and would be even less accurate when attempting to measure any changes to the lesion.

In some embodiments, VIT 100 may use augmented reality (AR) to provide accurate measurement across a range of magnifications. Crosshairs 112 may be arranged perpendicular to one another with their intersection proximal to the center of the lens thereby segmenting the magnified image into quadrants or subsections. One advantage illustrated by the use of these quadrants or subsections is to allow for close monitoring of skin lesions according to an established set of criteria.

The differentiated quadrants of VIT 100 are used as part of the VIM for the identification and early recognition of abnormal skin lesions. These allow for each skin lesion being examined to be quantified in the assessment of whether or not the lesion should be tested for cancer or even recommended for immediate removal. Magnifying lens 110 assists the medical professional in more easily identifying the features of the lesion, and the measurement reticles 122 help quantify the size of the lesion at the time of the assessment.

Although the embodiment shown in FIG. 1 includes four quadrants, any number of subsections may be used as long as the counting and assessment disclosed below are changed accordingly. For example, some embodiments may divide the magnified image into fifths, thirds, etc.

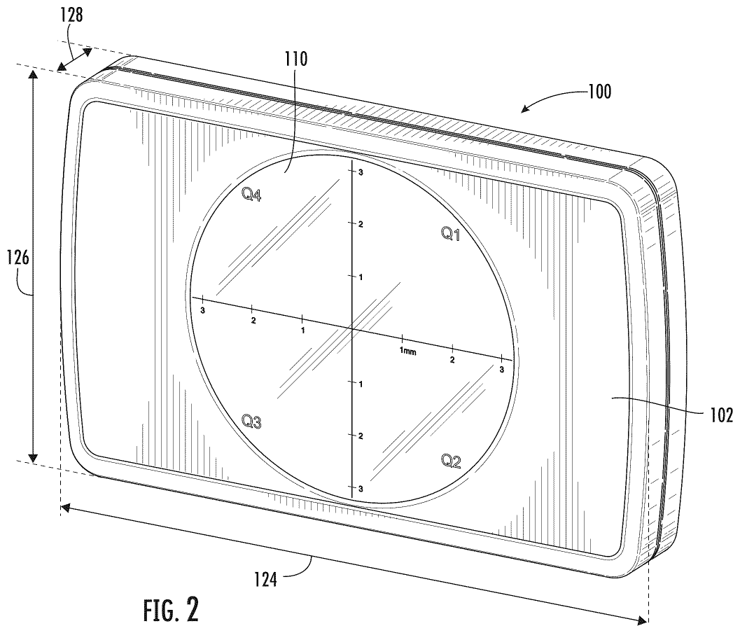

FIG. 2 shows a perspective view of VIT 100, shown in FIG. 1. FIG. 2 shows VIT 100's width 124, height 126, and depth 128. In some embodiments, width 124 may be approximately 118 mm, height 126 may be approximately 88 mm, and depth 128 may be approximately 15 mm.

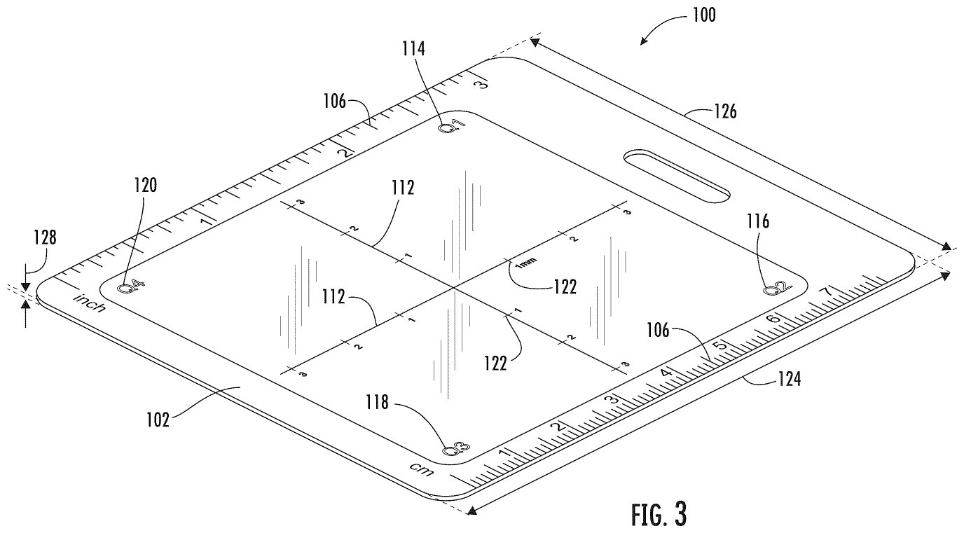

FIG. 3 shows a perspective view of VIT 100, according to some embodiments. This embodiment of VIT 100 has a compact form factor, similar to a credit card. To make magnifying lens 110 thinner, a Fresnel lens may be used. VIT 100 may have width 124 of 85.6 mm, height 126 of 53.98 mm, and depth 128 of 0.76 mm. VIT100 may also include additional measurement scales 106 to aid a medical professional in assessing lesion 144.

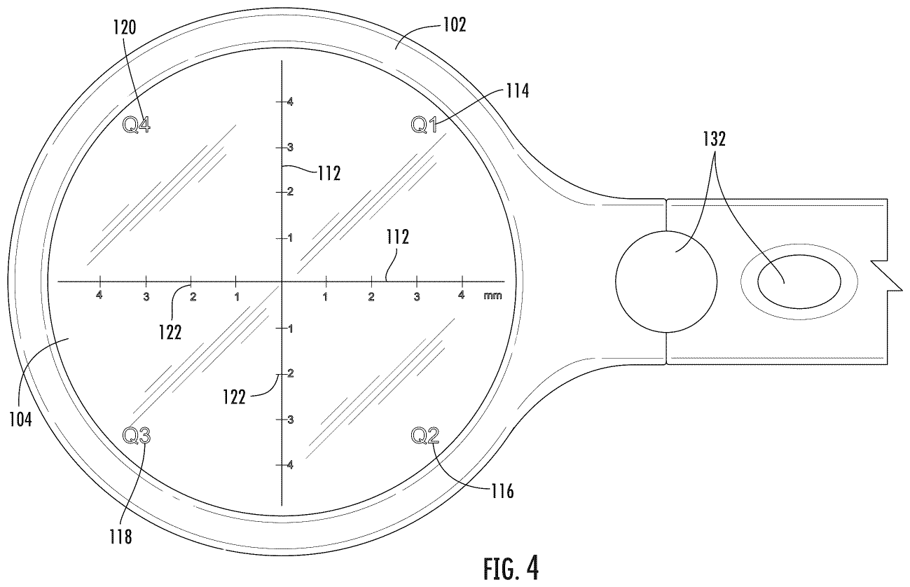

FIG. 4 shows a plan view of an embodiment of VIT 100. The embodiment shown in FIG. 4, has housing 102. Housing 102 is attached to handle 130. Handle 130 includes switches 132. Switches 132 may be configured to turn on a light to aid a medical professional in performing an assessment on a patient. In some embodiments, switches 132 may also control the level of magnification using the augmented reality described above.

VIT 100 may be used with visual inspection method (VIM). VIM recognizes clinical features not only for melanoma, but also non-melanoma skin cancers and atypical skin lesions at an early stage and may include multiple assessments. The VIT, as disclosed herein, assists the medical professional in rendering a more thorough evaluation according to the method as disclosed following. As part of the assessments according to the method, the abnormal lesion is compared to any potential changes to the normal baseline condition of the skin around it. For example, a first assessment may examine the changes in the number of border breaches, a second assessment may investigate the extent of color variations, and a third assessment may include changes in skin consistency. The method may comprise fewer than these assessments or more, depending on the presentation of the patient. These assessments are analyzed within each quadrant, and based on the analysis, a course of action is then recommended.

As part of performing a skin examination, the medical professional may first perform an aerial evaluation to visually identify unusual or abnormal lesions and to assess the patient's baseline skin characteristics. While taking an aerial view of the skin there can be raised lesions, skin tags, and different colored spots, any of which can be different or part of a person's normal-looking skin. For example, while conducting an aerial view of a patient's arm, freckles are noticed scattered on the skin, but there are no other different or suspicious marks. The skin located on the patient's arm would be considered normal-looking and can be used as their baseline condition. However, if there is a spot or lesion in the surrounding area that is different, new, or changing (abnormal) from the surrounding skin, a closer examination would be needed.

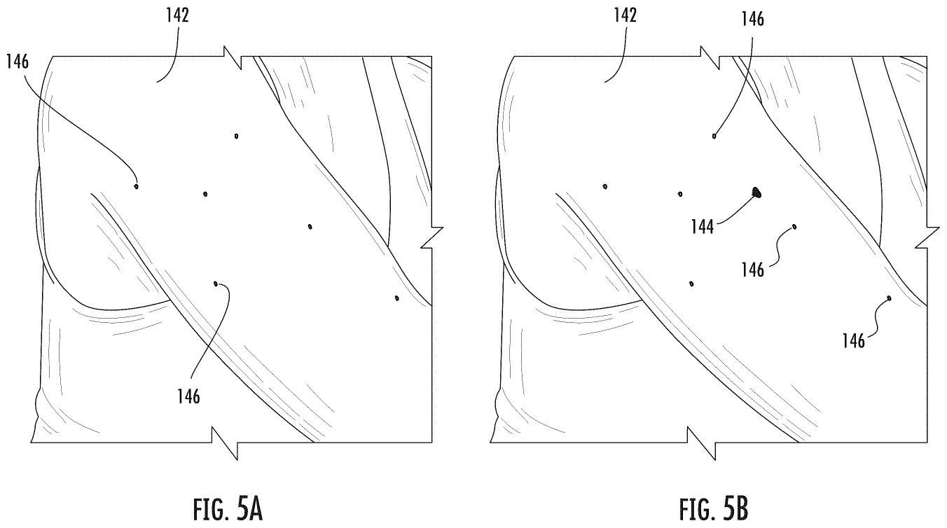

FIG. 5A shows patient 142. Patient 142 includes freckles 146. FIG. 5A is an illustration of the baseline skin of patient 142. FIG. 5B shows patient 142 with lesion 144. During the aerial evaluation, the medical professional would identify the unusual characteristics of lesion 144 in comparison to the normal or baseline skin of patient 142.

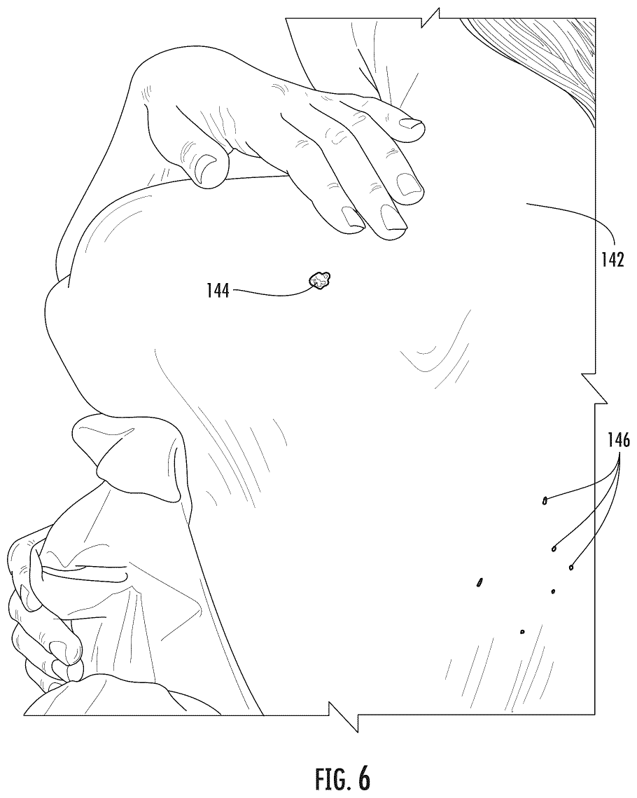

An aerial view of patient 142's back, showing lesion 144, is shown in FIG. 6. Lesion 144 may contain dark and light brown pigmented areas. This would be considered abnormal compared to the rest of patient 142's surrounding skin or baseline. Accordingly, further examination would be required.

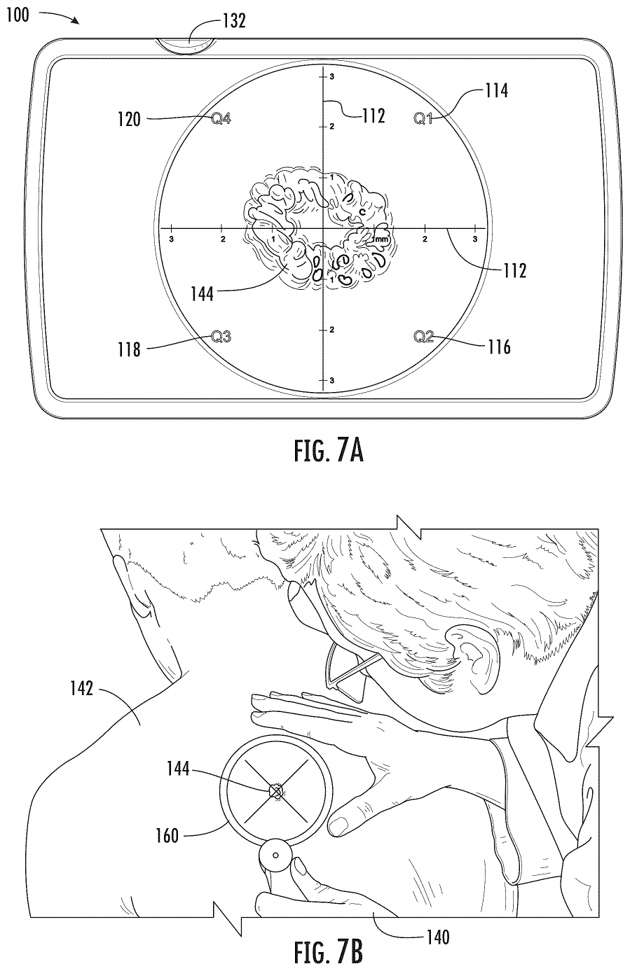

FIG. 7A shows VIT 100 being used to examine the skin of patient using the VIM method. VIT 100's crosshairs 112 are centered on lesion 144. As described above, crosshairs 112 divide the viewing area into four quadrants: first quadrant 114, second quadrant 116, third quadrant 118, and fourth quadrant 120. A portion of lesion 144 extends into each quadrant. FIG. 7B shows medical professional 140 using an embodiment of VIT 100 during the examination of patient 142's skin. As shown, medical professional 140 has centered VIT 100's magnifying lens 110 over lesion 144.

A method for identifying a skin lesion includes providing a VIT 100 as described above. VIT 100 includes magnifying lens 110 to produce a magnified image of an abnormal skin lesion 144. Magnifying lens 110 includes vertical and horizontal crosshairs 112 intersecting in a perpendicular configuration to segment the magnified image into quadrants. Each crosshairs 112 includes reticles 122.

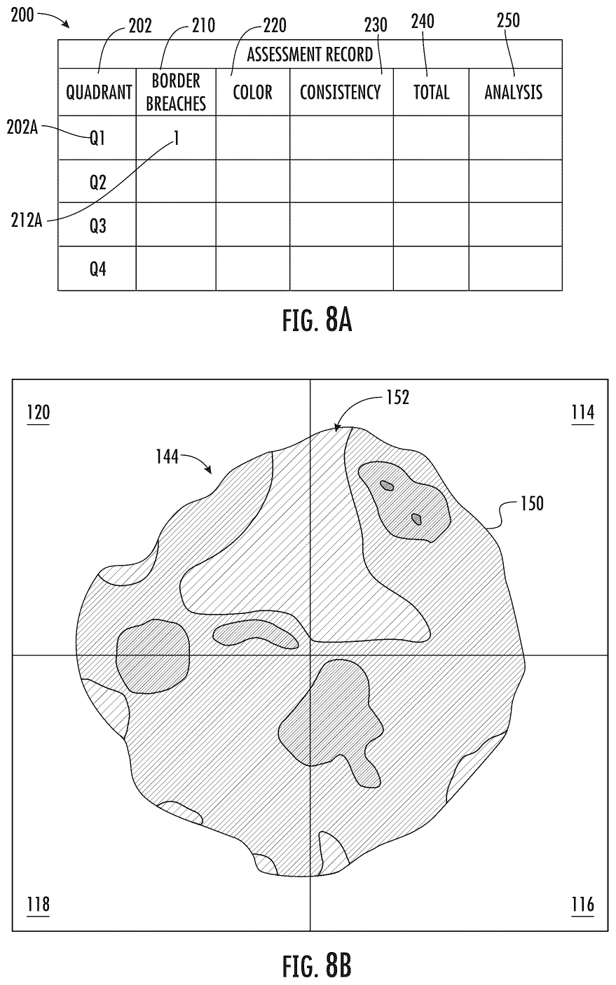

FIG. 8A shows an exemplary assessment record 200. Assessment record 200 includes rows 202 identifying each quadrant of the assessment. Additionally, the count of border breaches column 210, the count of color changes column 220, and consistency changes column 230 associated with each quadrant are identified in each column. Assessment record 200 also includes total column 240 and analysis column 250. Each portion of assessment record 200 will be described in further detail below.

The method includes performing a first assessment, which includes identifying, within first quadrant 114, an original border 150 (also referred to as a well-defined border) of lesion 144. The method continues with, using original border 150 as a comparator, counting, within first quadrant 114, each instance when a border of lesion 144 skin lesion breaches or encroaches in from the original border and recording, in record 212A, the number of instances where the border of lesion 144 breaches original border 150. For example, lesion 144 shown in FIG. 8B has original border 150 with one breach 152 of original border 150. Accordingly, in record 212A of assessment record 200, located at the intersection of border breach count column 210 and row 202A, the medical professional may record a “1,” as shown in FIG. 8A.

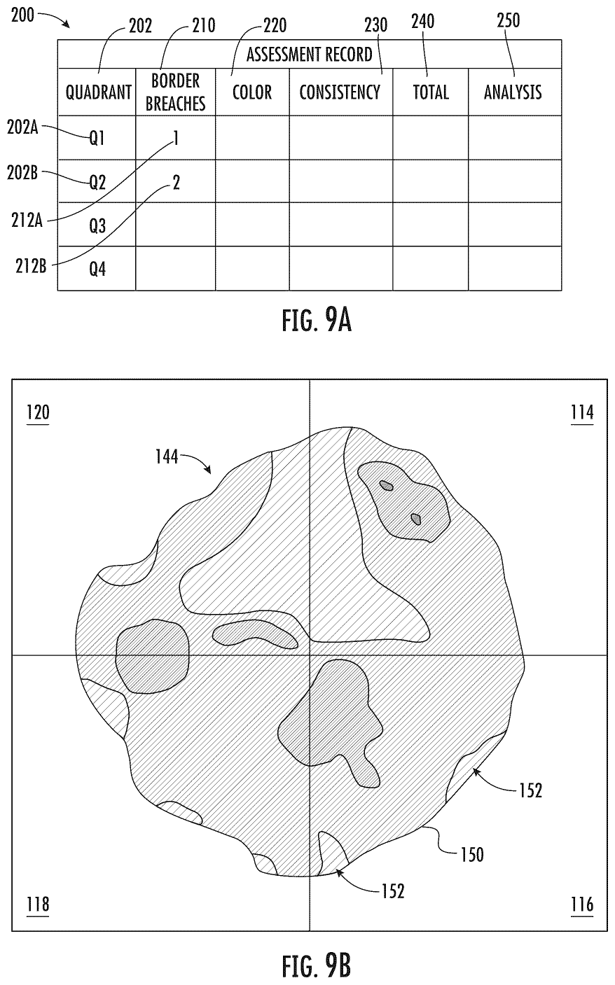

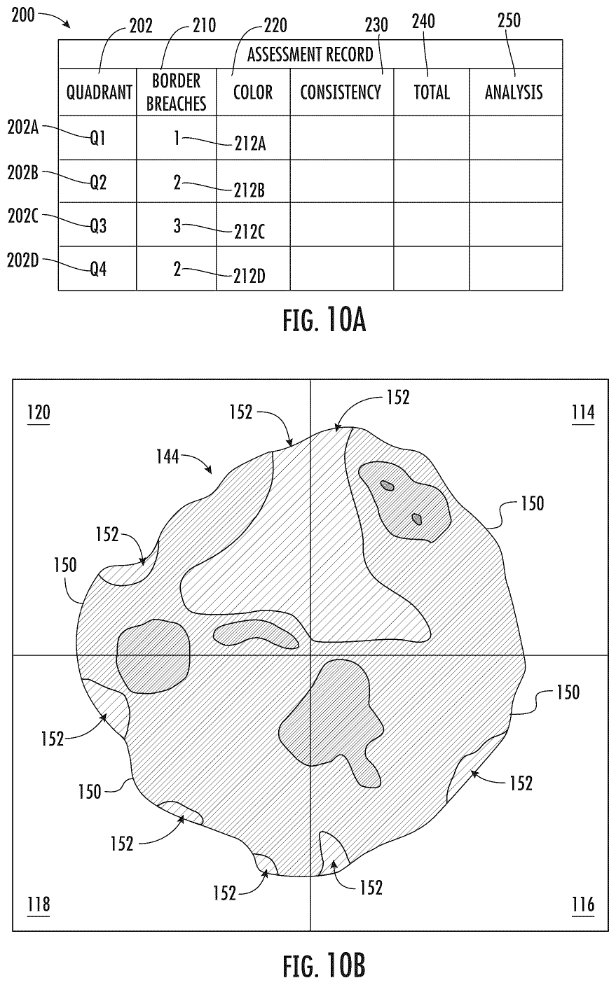

The method may further comprise repeating the first assessment for second quadrant 116, third quadrant 118, and fourth quadrant 120. FIG. 9A shows assessment record 200 with “2” shown in record 212B. This indicates that the portion of lesion 144 in second quadrant 116 has two breaches 152. Both breaches 152 are identified in FIG. 9B. FIG. 10A shows assessment record 200 with border breach count column 210 for each row 202A-D completed. FIG. 10B identifies exemplary border breaches 152. FIG. 10A shows that each record 212A-D includes the associated number of identified breaches 152 for each quadrant.

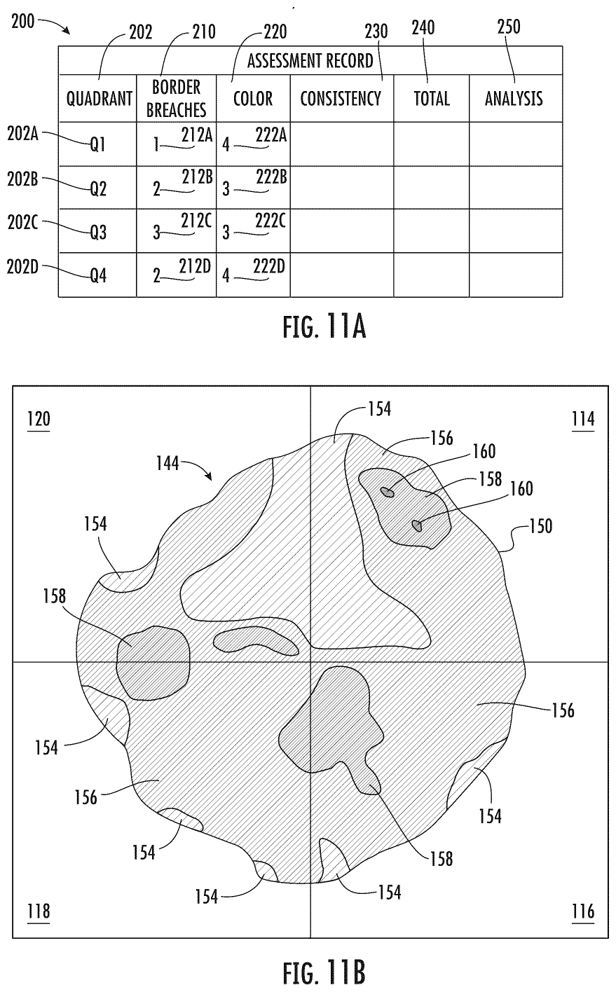

The method includes performing a second assessment, which includes identifying, within first quadrant 114, the number of color changes present in lesion 144 as compared to typical skin color variations present in the surrounding skin. FIG. 11A shows assessment record 200 with a completed color change column 220. FIG. 11A shows record 222A, which indicates that lesion 144 has “4” color changes in first quadrant 114. The method continues from first quadrant 114 to the other quadrants. Records 222B, 222C, and 222D indicate the number of color changes in lesion 144 in each of second quadrant 116, third quadrant 118, and fourth quadrant 120, respectively. FIG. 11B shows lesion 144 and identifies color changes. For example, lesion 144 may include first color 154, second color 156, third color 158, and fourth color 160. Each color may be a shade of a color. For example, first color 154 might be a light pink hue, second color 156 may be a light brown hue, third color 158 may be a medium brown, and fourth color 160 may be a dark brown.

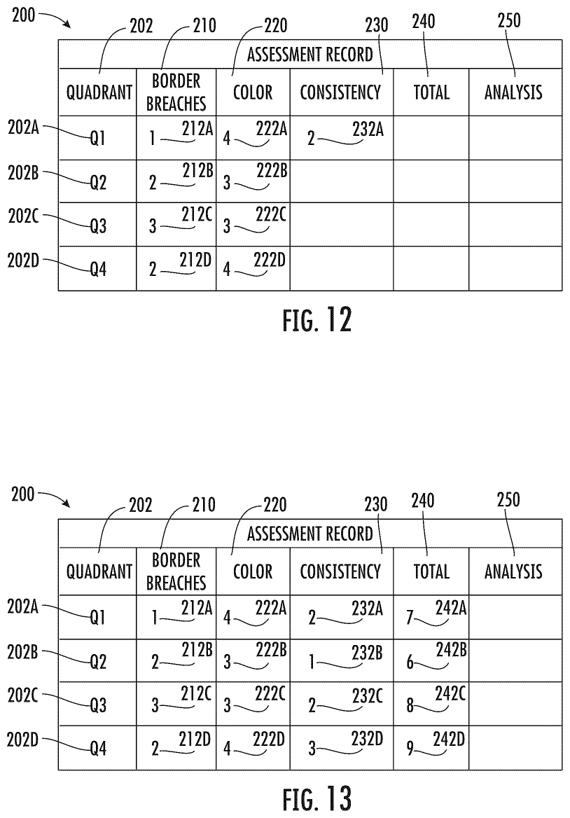

The method may also include performing a third assessment, which includes counting, within first quadrant 114, by either directly contacting the abnormal skin lesion or through visual observation using VIT 100, the number of consistency changes in the abnormal skin lesion within first quadrant 114. FIG. 12 shows assessment record 200 with “2” recorded in record 232A, which is in consistency column 230. This corresponds to the medical professional having determined that two consistency changes are present in first quadrant 114. The method continues with the medical professional assessing consistency changes in lesion 144 in each of the remaining quadrants.

In some embodiments, after determining the number of border breaches, color changes, and consistency changes in each quadrant for a lesion 144, a total column 240 for each quadrant may be determined. Total column 240 may be calculated by adding the values for each of the border breaches, color changes, and consistency changes for each of the quadrants and recording the sum in assessment record 200 as illustrated in FIG. 13. A total for each quadrant is calculated. First quadrant 114, recorded in row 202A, had one border breach (record 212A), four color changes (record 222A), and two consistency changes (record 232A) for a total of seven (record 242A). Second quadrant 116, recorded in row 202B, had two border breaches (record 212B), three color changes (record 222B), and one consistency change (record 232B) for a total of eight (record 242B). Third quadrant 118, recorded in row 202C, had three border breaches (record 212C), three color changes (record 222C), and two consistency changes (record 232C) for a total of eight (record 242C). Fourth quadrant 120, recorded in row 202D, had two border breaches (record 212D), four color changes (record 222D), and three consistency changes (record 232D) for a total of nine (record 242D).

Data collected as part of the method and compiled in assessment record 200 works in tandem with analysis rating scale 300 as part of the analysis phase. During the analysis phase, for each quadrant, the severity of the abnormal skin lesion is analyzed by comparing the total count from assessment record 200 with analysis rating scale 300 to assign a rating by the medical professional to each quadrant of the lesion 144.

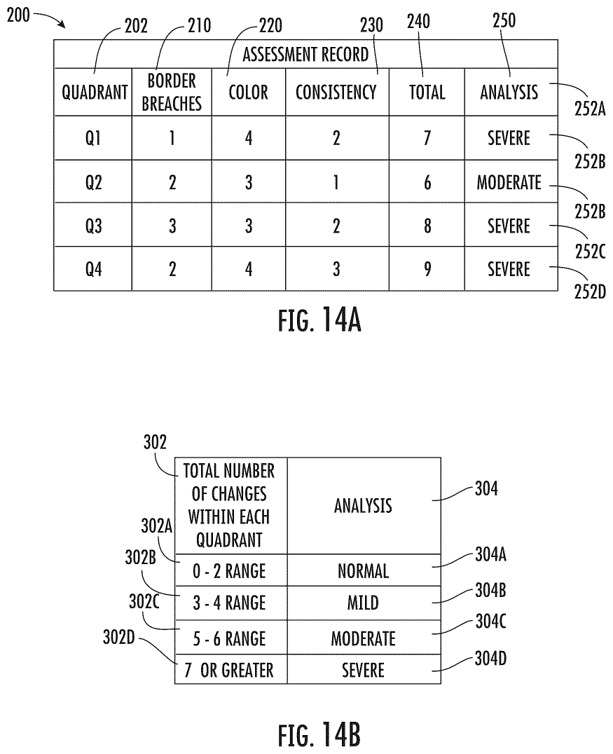

FIG. 14A shows completed assessment record 200. Analysis column 250 is completed by the medical professional by comparing the total for each quadrant (found in total column 240) to analysis rating scale 300's totals column 302 shown in FIG. 14B. Analysis rating scale 300 includes totals column 302 and analysis column 304. Totals column 302 identifies four ranges for totals determined for each quadrant in assessment record 200. Each range has a corresponding analysis. Range 302A corresponds to quadrants with a total of 0-2. This gives analysis 304A showing “Normal.” Range 302B corresponds to quadrants with a total of 3-4. This gives analysis 304B showing “Mild.” Range 302C corresponds to quadrants with a total of 5-6. This gives analysis 304C showing “Moderate.” Range 302D corresponds to quadrants with a total of 7 or greater. This gives analysis 304D showing “Severe.”

Analysis rating scale 300 is the result of extensive clinical research and relies on the identification of elements that go beyond the typical variations found in the skin. Because analysis rating scale 300 has been developed based upon extensive clinical research, it may provide a consistent and replicable source for a medical professional to assess abnormal skin lesions at an earlier stage than previously achieved. Thus, analysis rating scale 300 may provide a roadmap to general practitioners of definite, numerically based and unambiguous categorizations when presented with abnormal skin lesions.

Thereafter, the data and analysis are evaluated, and a course of action is recommended. If each of the four quadrants has a total count of 4 or lower and all quadrants were therefore analyzed as normal or mild, the entire abnormal lesion is evaluated and categorized as normal to mild, and immediate attention is not recommended. However, the patient is advised to keep a close watch on the lesion for any unusual changes and consult a physician annually. Taking a photo to document the lesion is also advised. Six months later, compare the photo of the abnormal lesion with the current abnormal lesion. In the event the lesion does change, a physician should be seen promptly for a thorough examination. If any one of the four quadrants has a total number of 5 or above and was analyzed as moderate or severe, the entire lesion is evaluated and categorized as moderate to severe. The patient is informed that the lesion necessitates prompt attention. The patient is advised to consult a physician for a thorough examination as soon as possible. When the abnormal skin lesions are categorized as moderate to severe, the physician may proceed to removal of the abnormal lesions.

The assessments according to the method as disclosed herein may be performed independently or in various combinations, and the total count and analysis rating scale adjusted accordingly. Additional assessments may be added and incorporated into the method as disclosed herein. While performing the method, it is preferable that an original rotational position of the VIT is maintained for subsequent measurements to maintain repeatability and consistency of results. In some embodiments, the method as disclosed herein may be performed on an image of an abnormal skin lesion for training purposes or in situations where the physician and the patient are not in person.

The disclosure further comprises a system for identification of skin lesions, including the disclosed VIT used according to the method as described above to produce the data (relating to the number of changes to the lesion within each quadrant) included within the assessment record. The assessment record may provide a substantially objective assessment of the severity of abnormal skin lesions, which is thereafter used in combination with the analysis rating scale, to analyze and rate an entire abnormal skin lesion and provide a subsequent recommendation to a patient. Using the system as disclosed herein, a more quantifiable and objective system is provided through which to assess any given lesion presented on a patient's body. The recommendation resulting from the analysis provided to the patient may be to take no action, follow up with in a specified time period such as 6 months, monitor the lesion ongoing for changes and if changes are observed, reassign an increased or weighted rating to the lesion, seek immediate care by a physician, and if the lesion is characterized as severe, recommend removal of the lesion. Thus, the substantially objective data within the assessment record (taken using the VIT according to the method) may result in a set of defined recommendations for follow up provided to the patient.

More specifically, this disclosure, its aspects, and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

It will be understood that implementations of the present disclosure include but are not limited to the specific components disclosed herein, as virtually any components consistent with the intended operation of the various disclosed embodiments may be utilized. Accordingly, for example, it should be understood that, while the drawings and accompanying text show and describe particular implementations, any such implementation may comprise any shape, size, style, type, model, version, class, grade, measurement, concentration, material, weight, quantity, and/or the like consistent with the intended operation of the disclosed implementations.

The concepts disclosed herein are not limited to the specific implementations or embodiments shown herein. For example, it is specifically contemplated that the components included in particular embodiments may be formed of any of many different types of materials or combinations that can readily be formed into shaped objects and that are consistent with the intended operation of the disclosure. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing.

Furthermore, the present disclosure may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular implementations, it should be readily apparent that a number of modifications may be made without departing from the spirit thereof and that these implementations may be applied to other implementations disclosed or undisclosed. The presently disclosed embodiments are, therefore, to be considered in all respects as illustrative and not restrictive.

What is claimed is:

1. A system for identification of skin lesion, comprising:
a handheld device comprising a magnifying lens configured to produce a magnified image of an abnormal skin lesion, wherein the magnifying lens comprises a vertical crosshair and a horizontal crosshair intersecting in a perpendicular configuration to segment the magnified image into quadrants, each crosshair having a linear scale disposed thereon;
placing the handheld device over an abnormal skin lesion of a patient;
locating the intersection of the crosshairs proximal to a center of the abnormal skin lesion;
performing a first assessment comprising:
identifying, within a first quadrant, an original border of the abnormal skin lesion;
counting, within the first quadrant, each instance when a border of the abnormal skin lesion breaches the original border; and
recording a number of instances where the border of the abnormal skin lesion breaches the original border within the first quadrant;
repeating the first assessment for a second, third, and fourth quadrant;
performing a second assessment comprising:
counting, within the first quadrant, a number of color changes within the abnormal skin lesion as compared to typical skin color variations present in the surrounding skin; and
recording the number of color changes within the first quadrant;
repeating the second assessment for the second, third, and fourth quadrant;
performing a third assessment comprising:
counting, within the first quadrant by way of direct contact with the abnormal skin lesion or through visual observation of the magnified image, a number of consistency changes in the abnormal skin lesion;
repeating the third assessment for the second through fourth quadrants;
calculating, for each quadrant, a total count resulting from the first, second and third assessments;
analyzing, for each quadrant, a severity of the of the abnormal skin lesion by comparing the total count with a rating scale;
assigning a rating to the abnormal skin lesion; and
based upon the ratings, recommending a course of action.

2. The system of claim 1, wherein the magnifying lens comprises a shape selected from the group consisting of a circular shape, an oval shape, an organic shape, a polygon shape, a rectangle shape, a square shape, and a geometric shape.

3. The system of claim 1 wherein the magnifying lens further comprises a reticle.

4. The system of claim 1, wherein the abnormal skin lesion is selected from the group consisting of a basal cell carcinoma, a squamous cell carcinoma, a Merkel cell carcinoma and a melanoma.

5. The system of claim 1, wherein the magnifying lens comprises an 8×-12× magnification.

6. The system of claim 1, wherein when the total count is calculated as 7 or greater, the abnormal skin lesion depicted within the quadrant is assigned a rating of severe.

7. The system of claim 1, wherein when the total count is calculated as from 5 to 6, the abnormal skin lesion depicted within the quadrant is assigned a rating of moderate.

8. The system of claim 1, wherein when the total count is calculated as from 3 to 4, the abnormal skin lesion depicted within the quadrant is assigned a rating of mild and no immediate action is taken.

9. The system of claim 1, wherein when the total count is calculated as from 0 to 2, the abnormal skin lesion depicted within the quadrant is assigned a rating of normal and no immediate action is taken.

10. The system of claim 1, wherein the system uses an image of the abnormal skin lesion.

11. The system of claim 1, wherein when each of the quadrants comprise a total count of 4 or less, the abnormal skin lesion as a whole is assigned a rating of normal or mild.

12. The system of claim 11, wherein based upon the assigned rating, a recommendation is provided to the patient to monitor the lesion for changes and follow up with a physician in 6 months if changes are observed.

13. The system of claim 12, wherein if changes are observed in the abnormal skin lesion during monitoring, the total count is increased and results in a weighted rating.

14. The system of claim 1, wherein when any one of the quadrants comprises a total count of 5 or greater, the abnormal skin lesion as a whole is assigned a rating of moderate or severe.

15. The system of claim 14, wherein based upon the assigned rating, a recommendation is provided to the patient to consult a physician for a thorough examination.

16. The system of claim 15, wherein based upon the assigned rating, the physician removes the abnormal skin lesion.

17. A handheld device comprising:
a magnifying lens having a magnification of about 8×-12×, configured to produce a magnified image of an abnormal skin lesion, the magnifying lens further comprising two crosshairs in a perpendicular configuration disposed thereon to segment the magnified image into equally sized quadrants, the crosshairs each having a linear scale disposed thereon wherein the linear scale is configured to measure in millimeters.

18. The handheld device of claim 17, further comprising a measurement reticle disposed on the magnifying lens, and wherein the measurement reticle comprises a linear scale which is modified as a function of the magnification of the magnifying lens.

19. The system of claim 17, wherein the linear scale is a function of a magnification of the lens.

20. The system of claim 17, wherein the rating is assigned to the abnormal skin lesion based upon a numerical quantification.
